(19) United States
(12) Patent Application Publication
(10) Pub. No.: US 2005/0113704 A1
(43) Pub. Date: May 26, 2005

(54) Patient monitoring system that incorporates memory into patient parameter cables

(76) Inventors: Corey J. Lawson, Sussex, WI (US); David George Hernke, Sussex, WI (US); Patrick Allen VanRyzin, Pewaukee, WI (US)

Correspondence Address: Patrick S. Yoder;FLETCHER YODER, P.O. Box 692289, Houston, TX, 77269-2289, US

(21) Appl. No.: 10/723,032

(22) Filed: Nov. 26, 2003

(51) Int. Cl. A61B 5/04

(52) U.S. Cl. 600/513

(57) ABSTRACT

A patient monitoring system having a patient parameter cable with a memory device is provided. The parameter cable having a memory device is capable of storing system information, such as configuration settings, patient-specific data, and cable identification information. Upon connection of the parameter cable to a subsequent monitoring station, the cable memory is read, and the station is configured in accordance with information stored in the parameter cable. Continuity of patient monitoring system settings and calibrations during disconnection and reconnection of patient parameter cables is maintained. Clinical workflow is improved. Opportunity for error that accompanies manual entry of patient data and information is reduced. Improved tracking capability facilitates quality control of parameter cables.

### BACKGROUND OF THE INVENTION

The present invention relates generally to the field of medical patient care, and more particularly, to the handling of data associated with patient monitoring systems.

Patient monitoring systems may incorporate a wide range of modality medical processes available to examine a patient's condition and health. Such modalities include, for example, electrocardiography/respiration (ECG/Resp), pulse oximetery (SpO₂), cardiac output (CO), temperature, invasive blood pressure (IBP), mainstream end tidal carbon dioxide (ETCO₂), non-invasive blood pressure (NBP), venous oxygen saturation (SvO₂), impedance cardiography (ICG), electroencephalography (EEG), Bispectral Index (BIS), neuromuscular transmission (NMT), entropy monitoring, spirometry/respiratory mechanics, metabolic monitoring, and anesthetic agent (i.e., gas) monitoring. Patient monitoring system components typically include a patient monitoring station, a parameter cable, one or more sensors, and so forth. The patient monitoring station may include a configurable or non-configurable acquisition device, an analytical device or machine, an operator workstation, user interface or display device, and the like. The parameter cable may connect the patient monitoring station to the sensors, which may be attached to a patient. The parameter cable, also called a trunk cable or signal acquisition cable, may be removable or detachable from the patient monitoring system.

One reason the parameter cables may be removable is to accommodate patient transport from one monitoring system or location to another. For example, detachable cables allow for the cable and sensors to remain attached to the patient while disconnecting the cable from a bedside monitoring system and reconnecting the cable to a transport monitoring system when transporting a patient to another care area of the same or different healthcare facility. Furthermore, upon arrival at the target care area, the cables may then be removed from the transport monitoring system and connected to a target bedside monitoring system. Patient parameter cables are commonly removed from the monitor station end and not at the patient end to minimize sensor manipulation, increase transition speed, and maintain patient comfort.

Another reason parameter cables may be detachable from a patient monitoring station is that the parameter cable is commonly a weak link in the patient monitoring system. As will be appreciated by those skilled in the art, hospital and other medical care providers regularly deal with parameter cable failures. Removal of the parameter cable from the patient monitoring station aids in troubleshooting and identification of a malfunctioning cable. On the other hand, the variety of cable manufacturers and the sheer number of cables utilized in the industry present difficulties in the tracking and quality control of parameter cables.

A problem with the removable parameter cables is that data is lost when the patient and cable are disconnected from one patient monitoring station and transferred to another patient monitoring station. System patient settings, calibrations, and patient data typically must be re-entered into the subsequent monitoring station upon reconnection of the parameter cable. During such a scenario, the clinician is commonly forced to manually re-enter patient specific information and re-perform necessary calibration procedures in order to maintain continuity of the patient record and optimal parameter accuracy. This is time consuming and detracts from clinical vigilance over the patient.

Patient parameter cables are integral to signal acquisition for typical patient monitoring systems and, as mentioned, are a high vulnerability component of patient monitoring systems. Existing capability to remove parameter cables from patient monitoring systems facilitates examination of the cables, but there is a need to improve tracking and quality control of parameter cables. Moreover, parameter cables are often disconnected from a patient monitoring station to facilitate patient transport and care, but patient/system data are lost. There is a need, at present, for the retention and continuity of system and patient data with patient monitoring systems having removable parameter cables.

### BRIEF DESCRIPTION OF THE INVENTION

The present technique promotes continuity of patient monitoring system settings and calibrations during disconnection and reconnection of patient parameter cables. Memory devices are disposed within the parameter cables, and thus, the parameter cables are capable of storing system information, such as configuration settings and patient-specific data, supplied by a first monitoring station. The memory is typically read/writable and may be used to store a variety of physiological patient parameters as well as factory-stored information, such as cable identification information. Upon connection of the parameter cable to a second monitoring station, the second monitoring station may read the cable memory and configure station fields and settings in accordance with information stored in the parameter cable. The present technique simplifies and improves speed of the preparation required before monitoring a patient, and thus improves clinical workflow. Furthermore, the technique reduces opportunity for error that accompanies manual entry of patient data and information. The correctness of data/information is essential to continuity of a patient record. Additionally, the present technique assists in the troubleshooting and quality control of parameter cables by providing for improved tracking capability.

With one aspect of the invention, a patient monitoring system includes a patient monitoring station, a parameter cable that connects the patient monitoring station to one or more sensors, and a memory device disposed within the parameter cable. The memory device may be disposed, for example, in a parameter cable adapter that connects the parameter cable to the patient monitoring station. On the other hand, the memory device may be disposed in a parameter cable adapter that connects the parameter cable to the one or more sensors. The memory device may be, for example, a one-wire memory chip. The patient monitoring station may incorporate one or more modalities of at least one of a electrocardiography/respiration (ECG/Resp), pulse oximetery (SpO₂), cardiac output (CO), temperature (Temp.), invasive blood pressure (IBP), mainstream end tidal carbon dioxide (ETCO₂), non-invasive blood pressure (NBP), venous oxygen saturation (SvO₂), impedance cardiography (ICG), electroencephalography (EEG), Bispectral Index (BIS), neuromuscular transmisssion, entropy monitoring, metabolic monitoring, anesthetic agent monitoring, and respiratory mechanics/spirometry. Additionally, a hardware device may be disposed on the patient monitoring station to facilitate communication between the patient monitoring station and the memory device.

Another aspect of the invention provides a patient parameter cable having a signal acquisition cable, an adapter that connects the signal acquisition cable to a patient monitoring station, and a memory device disposed in the adapter. The memory device may be, for example, a one-wire memory chip having a one-wire interface. Furthermore, the patient parameter cable may have one or more sensors and/or a sensor adapter that connects sensors to the signal acquisition cable.

The present invention also provides techniques for a patient parameter cable having a cable for signal acquisition, a station adapter for connecting the cable to a patient monitoring station, a sensor adapter for connecting the cable to one or more sensors, and a memory device disposed in the station adapter. Another patient parameter cable includes a cable for signal acquisition, a sensor adapter for connecting the cable to one or more sensors, and a memory device disposed in the sensor adapter. The patient parameter cable may also have a station adapter for connecting the cable to a patient monitoring station and/or one or more sensors connected to the sensor adapter.

Another facet of the present invention provides for a patient parameter cable that includes a cable for signal acquisition, a station adapter for connecting the cable to a patient monitoring station, a sensor adapter for connecting the cable to one or more sensors, and one or more memory devices disposed in at least one of the station adapter and sensor adapter. Furthermore, one or more sensors may be connected to the sensor adapter. Another patient parameter cable includes a cable for signal acquisition, a memory support disposed on the cable, and a memory device disposed in the memory support. This patient parameter cable may also include one or more sensors, a station adaptor, and/or a sensor adapter.

Yet another parameter cable, according to one aspect of the present invention, includes a cable for signal acquisition, a memory support disposed on the cable, a memory device disposed in the memory support, a station adapter for connecting the cable to a patient monitoring system, and a sensor adapter for connecting one or more sensors to the cable. The parameter cable may also include one or more memory devices stored in at least one of the station adapter and sensor adapter. The present technique also provides a patient parameter cable having means for carrying signals from one or more sensors to a patient monitoring station, means for connecting the parameter cable to a patient monitoring station, means for connecting the parameter cable to one or more sensors, and means for storing information in the parameter cable.

One aspect of the present invention gives a method for monitoring a patient that includes connecting a parameter cable having one or more sensors to a first patient monitoring station, affixing the one or more sensors to a patient, inputting demographics of the patient into the first patient monitoring station, calibrating the first patient monitoring station, monitoring the patient with the first patient monitoring station, and populating a memory device disposed in the parameter cable with demographics, calibration settings, and acquired monitored data. Furthermore, the method may include disconnecting the parameter cable from the first patient monitoring station, connecting the cable to a second patient monitoring station, retrieving the demographics, calibration settings, and acquired monitored data from the memory device into the second patient monitoring station, and monitoring the patient with the second patient monitoring station. Additionally, the method may also involve detaching and reattaching the one or more sensors to the patient. The first and second patient monitoring stations may incorporate, for example, modalities of at least one of a electrocardiography/respiration (ECG/Resp), pulse oximetery (SpO₂), cardiac output (CO), temperature (Temp.), invasive blood pressure (IBP), mainstream end tidal carbon dioxide (ETCO₂), non-invasive blood pressure (NBP), venous oxygen saturation (SvO₂), impedance cardiography (ICG), electroencephalography (EEG), Bispectral Index (BIS), neuromuscular transmission (NMT), entropy monitoring, metabolic monitoring, anesthetic agent monitoring, and respiratory mechanics/spirometry monitoring.

The present invention also provides techniques for a computer program, provided on one or more tangible media, for monitoring a patient, including a routine for populating a memory device disposed in a parameter cable with data from a first monitoring station. The computer program may also include a routine for retrieving the data from the memory device disposed in the parameter cable to a second monitoring station. The data may include at least one of a cable identification number, patient demographics, calibration settings, and analytical data.

### BRIEF DESCRIPTION OF THE DRAWINGS

FIG. 1 is an diagrammatical view of an exemplary patient monitoring system having a patient monitoring station, a patient parameter cable with a memory device disposed in an adapter that connects the cable to the station, and one or more sensors;

FIG. 2 is an diagrammatical of exemplary patient monitoring systems and associated mobility of the included patient parameter cable;

FIG. 3 is a diagrammatical view of an exemplary patient parameter cable having a memory disposed in an adapter that connects the cable to the patient monitoring station;

FIG. 4 is a diagrammatical view of an exemplary parameter cable adapter having a memory device;

FIG. 5 is a block diagram of an exemplary method for using a parameter cable having a memory device.

### DETAILED DESCRIPTION OF SPECIFIC EMBODIMENTS

Turning now to the drawings, and referring first to FIG. 1, a diagrammatical view of an exemplary patient monitoring system 10, is depicted. The patient monitoring system 10 may include a patient parameter cable 12 having a memory device 14 and connected to a patient monitoring station 16. The parameter cable 12 may join the patient monitoring station 16 to a patient 18 via one or more sensors 20 that attach to the patient 18. The one or more sensors 20 may be part of the cable 12 or may be a separate component, and/or may be detachable from the cable 12.

The parameter cable 12 may also include an adapter 22 that connects the cable 12 to the patient monitoring station 16, for example, at a plug 24. The plug 24 may represent a variety of interfaces that are complimentary to the connecting interface on the adapter 22. Additionally, the patient monitoring station 16 may comprise an acquisition device that receives data from the one or more sensors 20. The acquisition device may be configurable and may also facilitate communication between the patient monitoring station 16 and the memory device 14 disposed in the parameter cable 12.

The one or more sensors 20 may generate signals or data representative of the sensed parameters. Such raw data may be transmitted, for example, to the acquisition device in the patient monitoring station 16, which may acquire sampled or analog data, and may perform various initial operations on the data, such as filtering, multiplexing, and so forth. Further processing may be performed within the monitoring station 16, such as for additional filtering, analog-to-digital conversion, simple or detailed analysis of the data, and so forth. A display/user interface in the monitoring station 16 may permit the data to be manipulated, viewed, and output in a user-desired format, such as in traces on screen displays, hardcopy, and so forth. The particular configuration, of course, may vary depending upon the nature of the resource and the modality involved. Finally, the patient monitoring station 16 may be directly or indirectly linked to external systems and resources via a network link.

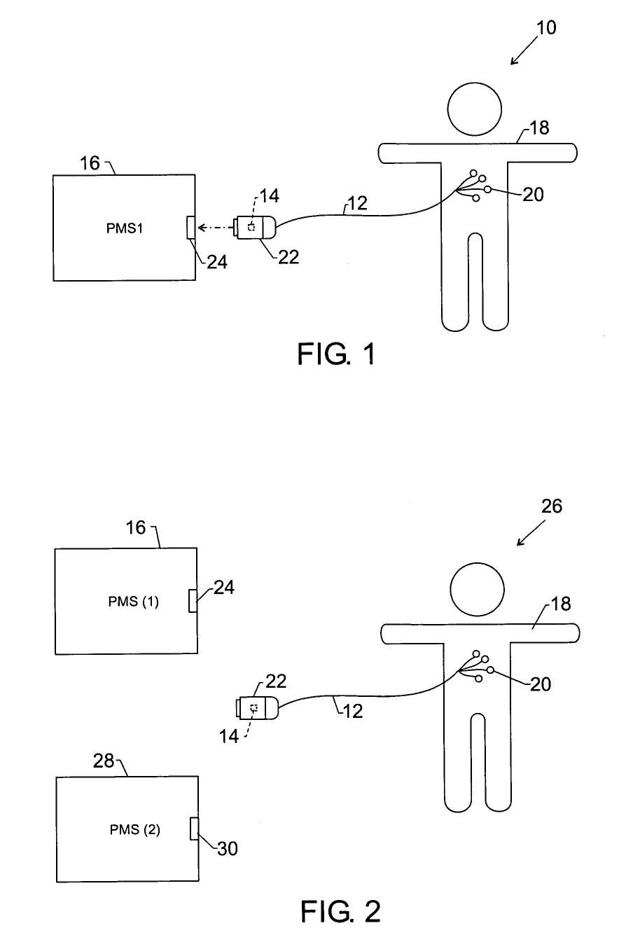

Referring to FIG. 2, a diagrammatical view of exemplary patient monitoring systems 26 is depicted. FIG. 2 is representative of two patient monitoring stations 16 and 28 sharing a mobile, removable parameter cable 12 having a memory 14. In this illustrative embodiment, the sensor 20 end of parameter cable 12 is attached to the patient 18, but the parameter cable 12 is not connected to either patient monitoring station 16 or 28. This configuration may occur, for example, when the patient 18 and cable 12 are in transfer between patient monitoring stations 16 and 28. In one scenario, the parameter cable 12 may initially be connected to the first monitoring station 16, and subsequently disconnected from the station 16 upon (or during) transfer of the patient 18. The parameter cable 12 may later be connected to the second monitoring station 28 during or after transfer, for example, via the adapter 22 at a plug 30. The parameter cable 12 (with memory device 14) may store system settings and patient information that can be retrieved by the second monitoring station 28 and subsequent monitoring stations.

It should be apparent that the present technique may accommodate more than two monitoring stations. The parameter cable 12 may be employed by multiple monitoring systems because system/patient information may be stored and retrieved by any number of patient monitoring stations. For example, a typical patient transport may utilize three monitoring systems: a first bedside monitoring system, a transport monitoring system, and a second bedside monitoring system. The parameter cable 12 may be initially connected to a bedside monitoring station, and then disconnected, and reconnected to a transport monitoring station for patient transport. After transport and upon reaching the target care area, the cable 12 may be disconnected, and reconnected to a second bedside monitoring station. The memory device 14 within the patient parameter cable 12 may be a read/writable device that stores/retrieves information for any number of monitoring systems.

A memory device 14, such as a 1-wire memory chip manufactured by Dallas Semiconductor (model no. DS2433), may be disposed in adaptor 22. The memory device 14 may store, for example, a cable identification number, patient demographic information, monitoring system settings and calibrations, acquired analytical data, and so forth. Modality types of the patient monitoring systems may include ECG/Resp, SPO₂, CO, temperature, IBP, ETCO₂, NBP, SvO₂, ICG, EEG, BIS, NMT, entropy monitoring, metabolic monitoring, anesthetic agent monitoring, spirometry/respiratory mechanics monitoring, and the like. Other suitable memory, processor, and communication circuits, chips, and chipsets may, of course, be employed in accordance with the present technique.

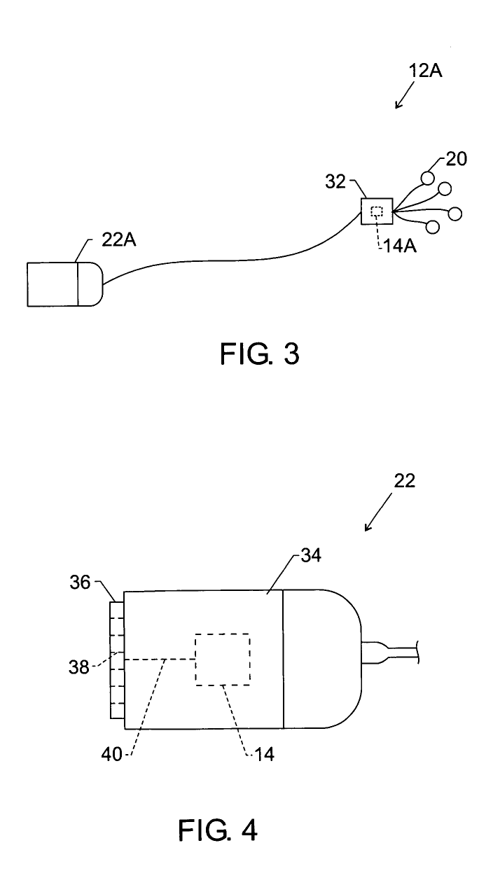

Referring to FIG. 3, a diagrammatical view of a parameter cable 12A, is depicted. A memory device 14A is disposed in a sensor connector 32 instead of the adapter 22A. It should be noted that the memory device 14 or 14A may be stored at any point along a parameter cable 12 or 12A, including in or on the trunk cable itself, or in the one or more sensors 20 (whether the sensors 20 or part of the parameter cable or not). It should be stressed that the present technique contemplates a wide range of configurations. For example, a memory device may be disposed in a memory support component installed on the parameter cable 12. Additionally, one or more memory devices may be disposed in a station adapter, sensor adapter, and/or memory support component. In general, the array of configurations contemplated by the present technique include, for example, parameter cables 12 having one or more combinations of memory devices, station adapters, sensor adapters, memory supports, sensors, and so forth.

Referring to FIG. 4, a diagrammatical view of an exemplary parameter cable adapter 22 used to connect the parameter cable 12 (FIG. 1) to a monitoring station 16 (FIG. 1), is depicted. A memory device 14, such as a 1-wire memory chip, is disposed within the adapter 22 having a body 34, interface 36, and pins 38. It should be noted that a variety of adapters 22 having different geometries and components may be used to connect the parameter cable 12 to a patient monitor station 16. In this illustrative embodiment, a single wire 40 connects the memory device 14 to one or more of the pins 38. The memory device 14 in this example is a 4096-bit 1-wire memory chip manufactured by Dallas Semiconductor (model DS2433).

With this exemplary memory chip, stored information may be accessed with minimal interface, for example, with a single port pin of a microcontroller or microprocessor. In other words, the control, address, data, and power may be reduced to a single data pin. The memory may be partitioned into sixteen 256-bit pages for packetizing data. A 255-bit scratch pad with strict read/write protocols may ensure integrity of data transfer. The memory chip may directly communicate through a port pin at up to 16.3 kbps. An overdrive mode may boost communication speed to 142 kps. An 8-bit family code may specify communication requirements to the reader with a presence detector acknowledging when the reader first applies voltage. Read and writes may occur over a wide voltage range, for example, over a range of 2.8 volts to 6.0 volts from −40° C. to +85° C. Again, however, such specific parameters and operational details will depend upon the circuitry or packaging selected.

The power to read and write the DS2433 is derived entirely from the 1-wire communication line. The memory is organized as sixteen pages of 256-bit each. The scratchpad is an additional page that acts as a buffer when writing to memory. Data is transferred serially via the 1-wire protocol which requires only a single data lead and a ground return. The DS2433 has three main data components: (1) 64-bit lasered ROM, (2) 256-bit scratchpad, and (3) 4096-bit Electrically Erasable Programmable Read-Only Memory (EEPROM). For power supply to the memory chip, a parasited-powered circuitry may obtain power whenever the I/O input is high, providing sufficient power based on the specified timing and voltage.

Here again, the present technique is not limited to any type of memory device. It should be apparent that the above discussion of the DS2433 is given only as an example, and that a wide variety of memory devices may be employed with the present technique.

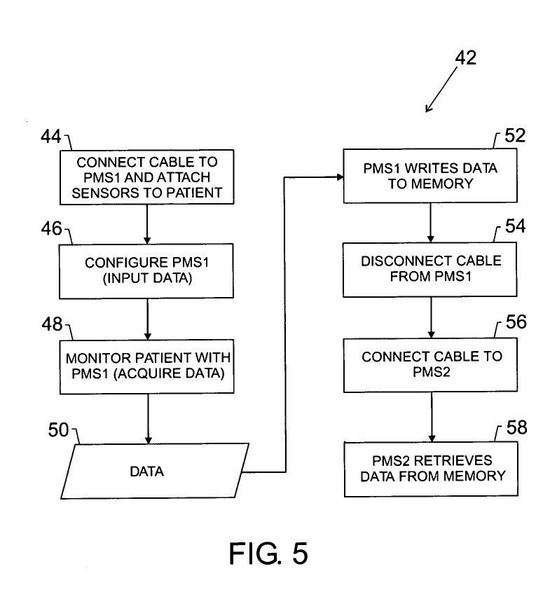

FIG. 5 is a block diagram of an exemplary method 42 for using a parameter cable 12 having a memory device 14. The exemplary method 42 may correlate with the two monitoring stations 16 and 28 of systems 26 illustrated in FIG. 2. Initially, the parameter cable 12 may be connected to a first patient monitoring station 16, and the sensors 20 attached to the patient 18 (block 44). The clinician and other users of the patient monitoring systems 26 (FIG. 2) may configure (block 46) the patient monitoring station 14, for example, by inputting patient demographic data, setting parameter limits, and calibrating the station. Parameter limits and calibration settings typically depend on the modality used and other circumstances of the analysis. Demographic data may include gender, age, race, ethnicity, disease prevalence, health risk factors, and so forth.

The first patient monitoring station 16 may monitor (block 48) the patient, acquiring analytical data. The first station 16 may populate (block 52) the memory device 14 with data 50, such as input data (block 46) and acquired data (block 48). In general, examples of stored data include patient demographics, primary patient ID, lead-set ID, cable ID, manufacturer ID, pace setting, alarm settings (limits and call levels), and sensor-type ID. Other examples include ST setting, trend scales setting, respiratory vector setting, respiratory apnea setting, spectrum setting, zero calibration setting, site ID setting, initial inflation pressure, and so forth. The type of data stored on the memory 14 may depend on the modality in use. Furthermore, protected data may pre-exist on the memory device 14, such as cable identification information stored during manufacture of the memory 14 or the cable 12.

A cable ID stored on the memory 14 in the parameter cable 12 may allow for better tracking of cable 12 failures. The cable ID, for example, may facilitate cross-referencing of the cable with other cables in similar monitoring systems, tracing of the cable to the upstream manufacture, comparison of the against other cables of the same manufacturing lot, maintaining cable information in a database, and so forth.

Continuing with the block diagram of FIG. 5, the parameter cable 12 may be disconnected (block 54) from the first patient monitoring station 16 and connected (block 56) to a second patient monitoring station 28. The second monitoring station 28 may retrieve (block 58) data 50 from the memory device 14 and populate the relevant fields of the second station. Thus, manual re-entry of data is minimized and continuity of system and patient data is enhanced. Moreover, as previously mentioned, the present technique may incorporate various modalities. In particular, the parameter cable 12 having a memory 14 may be utilized in a wide range of modalities. In conclusion, two exemplary modalities, the ECG and IBP, are discussed in more detail below. The two modalities are chosen for discussion purposes only.

Electrocardiography (EKG, ECG) is a technology that records electrical activity of the heart via electrodes (sensors 20) attached to a patient's 18 skin and coupled to a data acquisition system. The electrodes detect electrical impulses resulting from cardiac activity and do not apply electricity to the body. The electrical activity is detected, typically, through the skin on the chest, arms and legs of the patient 18 where the electrodes are placed. The technology application is typically performed by a specialized clinician, may take place in a hospital, clinic or laboratory. The electrodes used to detect the electrical activity, typically 3 or more, are placed at the desired locations via adhesive or other means.

The acquisition system of the monitoring station 16 (FIG. 1) translates the electrical activity as indicated by the impulses, into traces or lines. The ECG traces will typically follow characteristic patterns of the electrical impulses generated by the heart. Various parts of the characteristic pattern may be identified and measured, including portions of a waveform typically referred to as the P-wave, the QRS complex, the ST segment and the T-wave. These traces may be analyzed by a computer, nurse, or medical doctor for abnormalities which may be indicative of medical events or conditions. The ECG procedure is typically employed to identify such conditions as heart enlargement, signs of insufficient blood flow to the heart, signs of new or previous injury to the heart (e.g. resulting from heart attack), heart arrhythmias, changes in electrical activity of the heart caused by a chemical imbalance in the body, signs of inflammation of the pericardium, and so forth.

The present technique allows for the storing and continuity of the ECG data, even when, for example, a patient 18 is transported from one ECG monitoring station to another. Specific examples of data stored with the ECG modality may include patient demographics, primary patient ID, cable ID, manufacturer ID, lead-set ID, pace setting, alarm settings (limits and call levels), S setting, trend scales setting, respiratory vector setting, and respiratory apnea setting.

The noninvasive blood pressure (NBP) modality generally monitors blood pressure using an oscillometric technique. The oscillometric method of measuring blood pressure typically involves applying an inflatable cuff (sensor 20) around an extremity of a patient's 18 body, such as the patient's upper arm. The cuff is then inflated to a pressure above the patient's systolic pressure and then incrementally reduced in a series of small steps. A pressure sensor measures the cuff pressure at each step. The sensitivity of the sensor is such that pressure fluctuations within the artery resulting from the beating of the patient's heart may be detected. In particular, the pulses are transferred to the inflated cuff causing slight pressure variations within the cuff, which are detected by the pressure sensor. The pressure sensor produces an electrical signal based on the measured cuff pressure. The electrical signal comprises a DC component, representing the constant cuff pressure at the pressure step, and a series of small periodic components, representing the pressure variations attributable to the beating of the patient's heart. These small periodic components are often referred to as “oscillation complexes” or simply “oscillations.” A patient's 18 blood pressure may be estimated based on an analysis of these oscillation complexes.

Various waveforms may be generated that assist in analysis of the patient's 18 health and medical condition. Such waveforms may include a cuff pressure waveform that represents the overall pressure of the inflatable cuff at any given time, the arterial pressure waveform represented as a series of blood pressure pulses that indicate the periodic blood pressure variations corresponding to a patient's pulse, and a pressure oscillation curve comprising a series of cuff pressure oscillation complexes each of which is associated with a respective blood pressure pulse.

The present technique permits retention and continuity of NBP data when transferring the patient from one NBP station to another NBP station. Typical data that may be stored in the memory 14 in parameter cable 12 may include, for example, patient demographics, patient ID, cable ID, manufacturer ID, initial inflation pressure, alarm settings (limits and call levels), and trend scales setting.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A patient monitoring system comprising:
a patient monitoring station;
a parameter cable that connects the patient monitoring station to one or more sensors; and
a memory device disposed within the parameter cable.

2. The system of claim 1, wherein the memory device is disposed in a parameter cable adapter that connects the parameter cable to the patient monitoring station.

3. The system of claim 1, wherein the memory device is disposed in a parameter cable adapter that connects the parameter cable to the one or more sensors.

4. The system of claim 1, wherein the memory device is a 1-wire memory chip.

5. The system of claim 1, wherein the patient monitoring station comprises one or more modalities of at least one of a electrocardiography/respiration (ECG/Resp), pulse oximetery (SpO₂), cardiac output (CO), temperature (Temp.), invasive blood pressure (IBP), mainstream end tidal carbon dioxide (ETCO₂), non-invasive blood pressure (NBP), venous oxygen saturation (SvO₂), impedance cardiography (ICG), electroencephalography (EEG), Bispectral Index (BIS), Neuromuscular transmission (NMT), entropy monitoring, metabolics monitoring, anesthetic agent monitoring, and spirometry/respiratory mechanics monitoring.

6. The system of claim 1, wherein a hardware device disposed on the patient monitoring station facilitates communication between the patient monitoring station and the memory device.

7. A patient parameter cable comprising:
a signal acquisition cable;
an adapter that connects the signal acquisition cable to a patient monitoring station; and
a memory device disposed in the adapter.

8. The patient parameter cable of claim 7, wherein the memory device is a one-wire memory chip comprising a one-wire interface.

9. The patient parameter cable of claim 7, further comprising one or more sensors.

10. The patient parameter cable of claim 9, further comprising a sensor adapter that connects the one or more sensors to the signal acquisition cable.

11. A patient parameter cable comprising:
a cable for signal acquisition;
a station adapter for connecting the cable to a patient monitoring station;
a sensor adapter for connecting the cable to one or more sensors; and
a memory device disposed in the station adapter.

12. A patient parameter cable comprising:
a cable for signal acquisition;
a sensor adapter for connecting the cable to one or more sensors; and
a memory device disposed in the sensor adapter.

13. The patient parameter cable of claim 12, further comprising a station adapter for connecting the cable to a patient monitoring station.

14. The patient parameter cable of claim 12, further comprising one or more sensors connected to the sensor adapter.

15. The patient parameter cable of claim 14, further comprising a station adapter for connecting the cable to a patient monitoring station.

16. A patient parameter cable comprising:
a cable for signal acquisition;
a station adapter for connecting the cable to a patient monitoring station;
a sensor adapter for connecting the cable to one or more sensors; and
one or more memory devices disposed in at least one of the station adapter and sensor adapter.

17. The patient parameter cable of claim 16, further comprising one or more sensors connected to the sensor adapter.

18. A patient parameter cable comprising:
a cable for signal acquisition;
a memory support disposed on the cable; and
a memory device disposed in the memory support.

19. The patient parameter cable of claim 18, further comprising one or more sensors.

20. The patient parameter cable of claim 18, further comprising a station adapter.

21. The patient parameter cable of claim 18, further comprising a sensor adapter.

22. The patient parameter cable of claim 21, wherein one or more sensors are connected to the sensor adapter.

23. The patient parameter cable of claim 21, further comprising a station adapter.

24. A patient parameter cable comprising:
a cable for signal acquisition;
a memory support disposed on the cable;
a memory device disposed in the memory support;
a station adapter for connecting the cable to a patient monitoring system; and
a sensor adapter for connecting one or more sensors to the cable.

25. The parameter cable of claim 24, further comprising one or more memory devices stored in at least one of the station adapter and sensor adapter.

26. A patient parameter cable comprising:
means for carrying signals from one or more sensors to a patient monitoring station;
means for connecting the parameter cable to a patient monitoring station;
means for connecting the parameter cable to one or more sensors; and
means for storing information in the parameter cable.

27. A method for monitoring a patient comprising:
connecting a parameter cable having one or more sensors to a first patient monitoring station;
affixing the one or more sensors to a patient;
inputting demographics of the patient into the first patient monitoring station;
calibrating the first patient monitoring station;
monitoring the patient with the first patient monitoring station;
populating a memory device disposed in the parameter cable with demographics, calibration settings, and acquired monitored data.

28. The method of claim 27, further comprising:
disconnecting the parameter cable from the first patient monitoring station;
connecting the cable to a second patient monitoring station;
retrieving the demographics, calibration settings, and acquired monitored data from the memory device into the second patient monitoring station; and
monitoring the patient with the second patient monitoring station.

29. The method of claim 28, wherein the one or more sensors are detached and reattached to the patient.

30. The method of claim 28, wherein the first and second patient monitoring stations incorporate modalities of at least one of a electrocardiography/respiration (ECG/Resp), pulse oximetery (SpO₂), cardiac output (CO), temperature (Temp.), invasive blood pressure (IBP), mainstream end tidal carbon dioxide (ETCO₂), non-invasive blood pressure (NBP), venous oxygen saturation (SvO₂), impedance cardiography (ICG), electroencephalography (EEG), Bispectral Index (BIS), and neuromuscular transmission (NMT), entropy monitoring, metabolics monitoring, anesthetic agent monitoring, and spirometry/respiratory mechanics monitoring.

31. A computer program, provided on one or more tangible media, for monitoring a patient, comprising a routine for populating a memory device disposed in a parameter cable with data from a first monitoring station.

32. The computer program of claim 31 further comprising a routine for retrieving the data from the memory device disposed in the parameter cable to a second monitoring station.

33. The computer program of claim 32, wherein the data comprises at least one of a cable identification number, patient demographics, calibration settings, and analytical data.
